(19) United States
(12) Patent Application Publication
(10) Pub. No.: US 2026/0283797 A1
(43) Pub. Date: Sep. 24, 2026

(54) TRANSCATHETER ANNULOPLASTY SYSTEM AND METHODS

(71) Applicant: Valcare Medical, Inc., Wilmington, DE (US)

(72) Inventors: Nadav YELLIN, Aven Yehuda (IL); Yoav ROZEN, Binyamina (IL); Yuri FALKOVICH, Pardes Hana (IL)

(73) Assignee: Valcare Medical, Inc., Wilmington, DE (US)

(21) Appl. No.: 19/694,744

(22) Filed: Jun. 1, 2026

Related U.S. Application Data
(63) Continuation of application No. 19/363,425, filed on Oct. 20, 2025, now Pat. No. 12,642,654.
(60) Provisional application No. 63/709,798, filed on Oct. 21, 2024.

(51) Int. Cl. A61F 2/24

(52) U.S. Cl. CPC A61F 2/2448; A61F 2/2466; A61F 2220/0016; A61F 2230/0065

(57) ABSTRACT

Systems and methods are provided for an annuloplasty device and delivery system for treating valvular regurgitation. The annuloplasty device (e.g., a transcatheter annuloplasty ring) may include a tube having anterior and posterior anchors that may transition from a constrained state within the tube to a deployed state in which anchors extend through windows of the tube into tissue at the native valve site. Each anchor may include valvular and atrial anchors as well as anchors having different shapes (e.g., a curved anchor and a non-curved anchor). The annuloplasty device may be delivered to the native valve site via a catheter and the annuloplasty device may be manipulated within the patient's heart using a delivery handle to selectively deploy anchors while closing the anterior-to-posterior distance of the valve to treat valvular regurgitation.

### CROSS-REFERENCE TO RELATED APPLICATIONS

This application is a continuation of U.S. patent application Ser. No. 19/363,425, filed Oct. 20, 2025, now U.S. Pat. No. 12,642,654, which claims priority to U.S. Provisional Application No. 63/709,798, filed Oct. 21, 2024, the entire contents of each of which are incorporated herein by reference.

### TECHNICAL FIELD

This technology relates, in general, to an annuloplasty system and methods, for example, an annuloplasty system for transcatheter implantation of an annuloplasty ring at a native heart valve for the treatment of valvular regurgitation.

### BACKGROUND

Mitral Regurgitation is a valvular dysfunction that causes blood volume to flow during systole (during left ventricular contraction) from the left ventricle to the left atrium. In contrast, in a healthy heart, this direction of flow is blocked by the mitral valve. The reverse flow during systole causes pressure to rise in the left atrium, and maintaining a normal cardiac output results in an increased pressure in the left ventricle.

Treating patients with MR (mitral regurgitation) or TR (tricuspid regurgitation) could require valve replacement in order to reduce or eliminate the regurgitation. For many years, the commonly accepted treatment was surgical repair or replacement of the native valve during open heart surgery. In recent years, a transcatheter technique has been developed for introducing and implanting a prosthetic heart valve using a flexible catheter in a manner that is less invasive than open heart surgery with fewer risks of complications.

In the transcatheter technique, a prosthetic device is delivered to the native valve site (e.g., aortic valve, mitral valve, or tricuspid valve) through a catheter while the device is constrained in a shaft with a reduced diameter. The device is then expanded when it is delivered to the correct position at the native valve site. Examples of such prosthetic devices for treating valvular regurgitation, and related processes for delivering the prosthetics through a catheter, are described in U.S. Pat. No. 8,518,107 to Tsukashima et al., U.S. Pat. No. 10,543,087 to Yellin et al., U.S. Pat. No. 11,103,349 to Yellin et al., U.S. Pat. No. 11,534,300 to Yellin et al., U.S. Pat. No. 11,576,779 to Yellin et al., U.S. Pat. No. 11,806,237 to Rozen et al., U.S. Pat. No. 11,857,418 to Yellin et al., the entire contents of each of which are incorporated herein by reference.

Advancing the catheter to the target site can be achieved through: (a) the vascular system where a catheter is advanced from the femoral vein/artery, or any other blood vessel that allows access to the target site; (b) trans-apically where a catheter is advanced through a small incision made in the chest wall and then through the apex; or (c) trans-atrially where a catheter is advanced through a small incision made in the chest wall and then through the left or right atrium, for example.

While valves and other prosthetic devices are capable of transcatheter delivery and implantation at the native valve site, transcatheter devices for treating valvular regurgitation often are difficult to secure to the native valve tissue. More specifically, transitioning anchors from a constrained state to a deployed state during transcatheter delivery in a manner sufficient to firmly secure the prosthetic at the native valve site is a significant challenge. Additionally, reducing the anterior-posterior distance of the native valve during transcatheter implantation of the prosthetic has proven to be difficult due the constraints of transcatheter delivery.

Accordingly, there is a need for improved annuloplasty devices, systems, and methods for treating valvular regurgitation having enhanced features for securing the annuloplasty device to the native valve and reducing the anterior-posterior distance of the valve.

### SUMMARY

Provided herein are systems and methods for an annuloplasty device and delivery system for treating valvular regurgitation. The annuloplasty device (e.g., a transcatheter annuloplasty ring) may include a tube having anterior and posterior anchors having anchors with different shapes (e.g., curved anchors and a linear and/or non-curved anchor). The anterior and posterior anchors may transition from a constrained state within the tube to a deployed state in which anchors extend through windows of the tube into tissue at the native valve site to secure the annuloplasty device to the native valve. Each anchor may include valvular and atrial anchors such that the annuloplasty device may be secured to both valvular and atrial tissue. The annuloplasty device may be delivered to the native valve site via a catheter and the annuloplasty device may be manipulated within the patient's heart to selectively deploy anchors to close the anterior-to-posterior distance of the valve thereby treating valvular regurgitation.

An annuloplasty device is provided herein and may includea tube having a first end, a second end, an inner lumen, a first plurality of windows, and a second plurality of windows, the first plurality of windows designed to be orientated toward anterior tissue of a native valve and a second plurality of windows designed to be orientated toward posterior tissue of the native valve when the annuloplasty device is implanted at a native valve site, at least one anterior anchor having a first elongated base, a first plurality of anchors extending from the first elongated base, each anchor of the first plurality of anchors having a first shape, and at least one first anchor extending from the first elongated base and having a second shape different than the first shape, the at least one anterior anchor designed to transition from a first constrained state fully disposed within the tube to a first deployed state in which the first plurality of anchors and the at least one first anchor extend through at least some of the first plurality of windows into the anterior tissue of the native valve, at least one posterior anchor having a second elongated base, a second plurality of anchors extending from the second elongated base, each anchor of the second plurality of anchors having a third shape, and at least one second anchor extending from the second elongated base and having a fourth shape different than the third shape, the at least one posterior anchor designed to transition from a second constrained state fully disposed within the tube to a second deployed state in which the third plurality of anchors and the at least one second anchor extend through at least some of the second plurality of windows into posterior tissue of the native valve.

The annuloplasty device may include a second anterior anchor including a third elongated base, a third plurality of anchors extending from the third elongated base, each anchor of the third plurality of anchors having the first shape, and at least one third anchor extending from the third elongated base and having the second shape, the second anterior anchor designed to transition from a third constrained state fully disposed within the tube to a third deployed state in which the third plurality of anchors and the at least one third anchor extend through at least some of the first plurality of windows into the anterior tissue of the native valve, a second posterior anchor having a fourth elongated base, a fourth plurality of anchors extending from the fourth elongated base, each anchor of the fourth plurality of anchors having the third shape, and at least one fourth anchor extending from the fourth elongated base and having the fourth shape, the fourth posterior anchor designed to transition from a fourth constrained state fully disposed within the tube to a fourth deployed state in which the third plurality of anchors and the at least one fourth anchor extend through at least some of the second plurality of windows into posterior tissue of the native valve.

The first plurality of anchors in the first deployed state and the third plurality of anchors in the third deployed state may be axially aligned with respect to the tube and offset from one another such that the first plurality of anchors in the first deployed state does not interfere with the third plurality of anchors in the third deployed state. The tube may further include a locking window designed to prevent the at least one anterior anchor from transitioning from the first constrained state to the first deployed state. The first elongated base may include a first void biased to reduce in size and a protrusion adjacent to the first void including a first portion and a second portion separated by a second void, and wherein the anterior anchor further may include a pulley designed to move within the first void from a first position designed to cause the first void to expand in size to a second position designed to permit the first void to reduce in size.

When the pulley is in the first position, the first and second portions of the protrusion may be caused to extend into the locking window of the tube and to move apart from one another and, when the pulley is in the second position, the first and second portions of the protrusion may be caused to retract from the locking window and move toward one another permitting the pulley to move with respect to the tube thereby causing the first plurality of anchors to extend through at least some of the plurality of windows and into tissue of the native valve site.

The annuloplasty device may further include a pulley activator in mechanical communication with the pulley and a handle positioned extracorporeally and designed to selectively transition the pulley from the first position to the second position. The at least one first anchor may be substantially linear and is positioned at an end of the elongated base. The annuloplasty device may include a lock having a first engagement designed to engage the first end with the second end and a second engagement designed to selectively disengage the first end and the second end. The tube may include an inner layer having a polymer and an outer layer having a fabric.

Yet another annuloplasty device is provided herein having a tube having an inner lumen, a plurality of windows, and a locking window, the tube designed to form a ring shape upon delivery to a native valve site of a patient, at least one anchor including an elongated base, a plurality of anchors extending from the elongated base, a first void biased to reduce in size, and a protrusion adjacent to the first void and including a first portion and a second portion separated by a second void, and a pulley designed to move within the first void from a first position designed to cause the first void to expand in size to a second position designed to permit the first void to reduce in size, wherein, when the pulley is in the first position, the first and second portions of the protrusion are caused to extend into the locking window of the tube and to move apart from one another and, when the pulley is in the second position, the first and second portions of the protrusion are caused to retract from the locking window and move toward one another permitting the pulley to move with respect to the tube thereby causing the plurality of anchors to extend through at least some of the plurality of windows and into tissue of the native valve site.

The base of the protrusion may include an indention designed to receive a portion of the pulley and to resist movement of the pulley from the first position to the second position. Each anchor of the plurality of anchors has a first shape, and the at least one anchor may further include at least one secondary anchor extending from the first elongated base and having a second shape different than the first shape. The at least one secondary anchor may be positioned at an end of the end of at least one anchor. The first shape may be curved and the second shape may be substantially linear.

The annuloplasty device may further include at least one second anchor including a second elongated base, a plurality of second anchors extending from the second elongated base, a third void biased to reduce in size, and a second protrusion adjacent to the third void and include a third portion and a fourth portion separated by a fourth void, and a second pulley designed to move within the third void from a third position designed to cause the third void to expand in size to a fourth position designed to permit the third void to reduce in size.

The tube may further include a second locking window and wherein, when the second pulley is in the third position, the third and fourth portions of the protrusion are caused to extend into the second locking window of the tube and to move apart from one another and, when the pulley is in the fourth position, the third and fourth portions of the protrusion are caused to retract from the second locking window and move toward one another permitting the second pulley to move with respect to the tube thereby causing the plurality of second anchors to extend through at least some of the plurality of windows and into tissue of the native valve site.

When the pulley is in the second position, the at least one anchor may be designed to enter anterior tissue of the native valve site and, when the second pulley is in the fourth position, the at least one second anchor is designed to enter posterior tissue of the native valve site. The tube may further include a first end and a second end, the annuloplasty device may further include a lock having a first engagement designed to engage the first end with the second end and a second engagement designed to selectively disengage the first end and the second end. The tube may include an inner layer including a polymer and an outer layer including a fabric.

The foregoing summary is illustrative only and is not intended to be in any way limiting. In addition to the illustrative aspects, embodiments, and features described above, further aspects, embodiments, and features will become apparent by reference to the following drawings and the detailed description.

### BRIEF DESCRIPTION OF THE DRAWINGS

FIG. 1 shows exemplary components of an annuloplasty system for delivering an annuloplasty device for treating valvular regurgitation including a catheter system and an annuloplasty device.

FIG. 2 illustrates a perspective view of an annuloplasty device having anterior and posterior anchors, each with atrial and ventricular anchors.

FIGS. 3A-3C illustrate side and top views of an annuloplasty device having anterior and posterior anchors, each with atrial and ventricular anchors.

FIG. 4 illustrates an exploded perspective view of an annuloplasty device having two posterior anchors and two anterior anchors.

FIGS. 5A-5E illustrate perspective views and side views of an anterior anchor, pulley, and pulley activator of an annuloplasty device.

FIGS. 6A-6C illustrate perspective views and side views of a posterior anchor and pulley of an annuloplasty device.

FIGS. 7A-7C illustrate perspective views and cut-away views of a track anchor and a dock of an annuloplasty device.

FIGS. 8A-8G illustrate perspective views deploying track anchors, posterior anchors, and anterior anchors of an annuloplasty device.

The foregoing and other features of the present invention will become apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings.

### DETAILED DESCRIPTION

Provided herein are systems and methods for annuloplasty devices for treating valvular regurgitation. For example, annuloplasty systems may include a catheter system for minimally invasive percutaneous procedures for implanting an annuloplasty device (e.g., an annuloplasty ring) for the treatment of mitral and/or tricuspid regurgitation. The catheter system may include a delivery handle for manipulating the annuloplasty device, which may be positioned in an elongated catheter, delivering the annuloplasty device to a target site of the native valve, aligning the implant with the anatomy to create intimate contact between the annuloplasty device and the anatomy, releasing the annuloplasty device at the target site, and safely retrieving the delivery system.

The annuloplasty device may be used to treat valvular regurgitation and may be formed into a ring shape for implantation at the native valve site (e.g., mitral, tricuspid, etc.). The annuloplasty device may include a tube, which may be a multi-layered tube having multiple windows, one or more posterior anchors and one or more anterior anchors, which each may be selectively transitioned from a constrained positioned within the tube to a deployed positioned extending through the windows and into tissue of the native valve. The annuloplasty device may be delivered to a native valve site in an elongated state and caused to form a ring shape upon reaching the native valve. Track anchors may extend from the distal end of the catheter system and may be used together with the posterior and anterior anchors to reduce an anterior-posterior distance of the native valve to reduce valvular regurgitation.

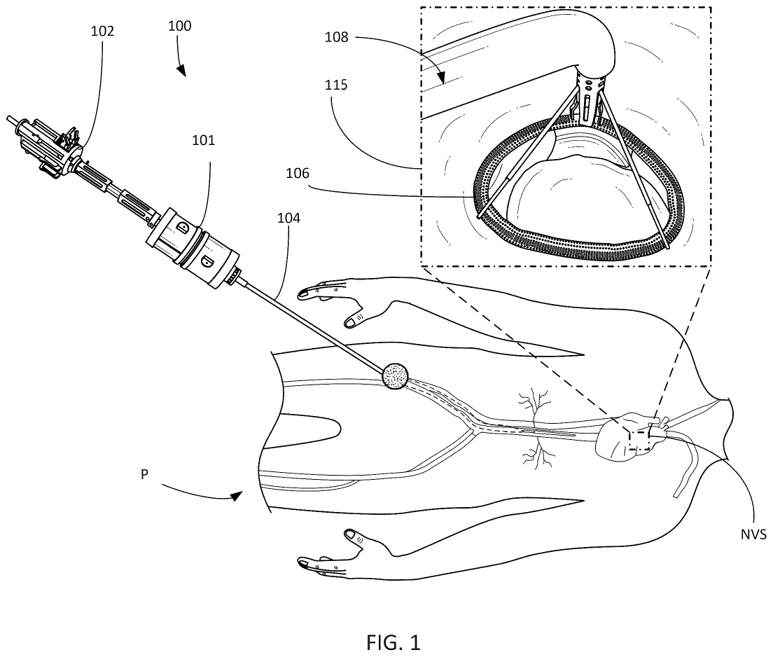

Referring now to FIG. 1, annuloplasty system 100 is illustrated. Annuloplasty system 100 may be used to deliver annuloplasty device 106 to the native valve site NVS (e.g., illustratively the mitral valve) of the patient P. As shown in FIG. 1, annuloplasty system 100 may include catheter system 101 which may include delivery handle 102 and elongated catheter 104, which may extend from delivery handle 102 and may have a distal end 108 that may be inserted into the patient P, for example at the femoral artery. Distal end 108 may be guided to a native valve site at an individual's heart, which may be the mitral valve or any other native valve (e.g., tricuspid valve) experiencing valvular regurgitation. It will be understood by one of ordinary skill in the art that distal end 108 may be introduced to the individual's vasculature at another suitable location (e.g., groin, thigh, stomach, chest, neck, or collar bone, etc.).

Catheter system 101 of annuloplasty system 100 may be similar to the catheter system described in U.S. Pat. No. 10,543,087, the entire contents of which are incorporated herein by reference. Delivery handle 102 may include multiple knobs, actuators, and/or other features for manipulating elongated catheter 104 and/or distal end 108 of the catheter. For example, delivery handle 102 may include one or more knobs and/or actuators for guiding distal end 108 through the patient's vasculature to the native valve site and/or may include multiple knobs and/or actuators for selectively deploying track anchors, posterior anchors, and/or anterior anchors of annuloplasty device 106 to deploy annuloplasty device 106 shown in the close-up view at the native valve site NSV.

Using delivery handle 102, a user of annuloplasty system 100 may deliver annuloplasty device 106 in an elongated state with one end disconnected from another end, and may cause annuloplasty device 106 to form a ring and/or annular shape, as shown in FIG. 1, upon entering the heart. For example, upon entering the left atrium, delivery handle 102 may manipulate annuloplasty device 106 to connect the two ends of annuloplasty ring 106. Knobs and/or actuators in mechanical communication with annuloplasty device 106 and/or distal end 108 may then be manipulated to sequentially release anchors from the posterior and anterior portions of annuloplasty device 106 to close the anterior-posterior gap of the native valve to treat valvular regurgitation.

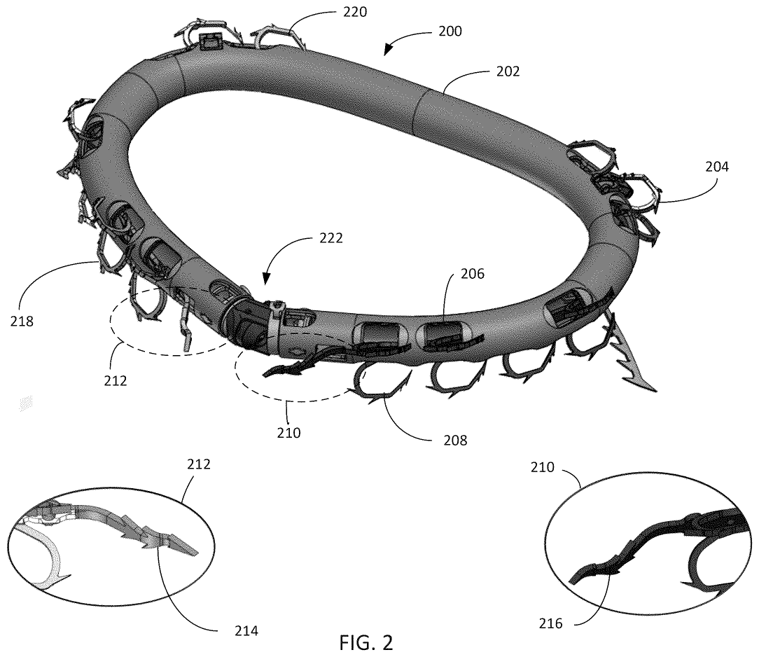

Referring now to FIG. 2, a perspective view of an annuloplasty device having anterior and posterior anchors, each with atrial and ventricular anchors, is illustrated. As shown in FIG. 2, annuloplasty device 200, which may be the same as or similar to annuloplasty device 106 of FIG. 1, may include tube 202 which may have multiple layers and several windows 206. Windows 206 may extend through an outer layer of tube 202 to an inner lumen of tube 202. Windows 206 may include windows oriented towards an anterior side of annuloplasty device 200, designed to be placed adjacent to anterior tissue of a native valve, as well as a posterior side of annuloplasty device 200, designed to be placed adjacent to posterior tissue of a native valve. Tube 202 may be connected at either end via closure 222 which may include a closing engagement for securing both ends of tube 202 and a release engagement for disengaging each end of tube 202.

Annuloplasty device 200 may include one or more anterior anchors for extending to anterior tissue of a native valve and one or more posterior anchors for extending into posterior tissue to secure annuloplasty device 200 to the anterior and posterior tissue. For example, annuloplasty device 200 may include anterior anchor 220 and anterior anchor 204, which may be transitioned from a constrained state inside of the inner lumen of tube 202 to a deployed state with anchors extending through at least some of windows 206 of tube 202 into anterior tissue of the native valve.

Annuloplasty device 200 may further include posterior anchors 208 and 218, which may be transitioned from a constrained state inside of the inner lumen of tube 202 to a deployed state with anchors extending through at least some of windows 206 of tube 202 into posterior tissue of the native valve. As shown in view 210, locking anchor 216 of posterior anchor 208 may extend towards posterior anchor 218 and similarly, as shown in view 212, anchor 214 of posterior anchor 218 may extend towards posterior anchor 208. Locking anchors 214 and 216 may be serrated and may be designed to secure the ends of annuloplasty device 200 into the tissue of native valve.

Each of anterior anchors 204 and 220 and posterior anchors 208 and 218 may include two rows of curved anchor portions, with one row for insertion into ventricular tissue and another row for insertion into atrial tissue. Each of anterior anchors 204 and 220 and posterior anchors 208 and 218 may further include a non-curved anchor, a linear anchor, and/or an anchor with a shape different than the curved anchors. Each of anterior anchors 204 and 220 and posterior anchors 208 and 218 may be serrated and/or have barbs or hooks for piercing and securing such anchors to tissue of the native valve.

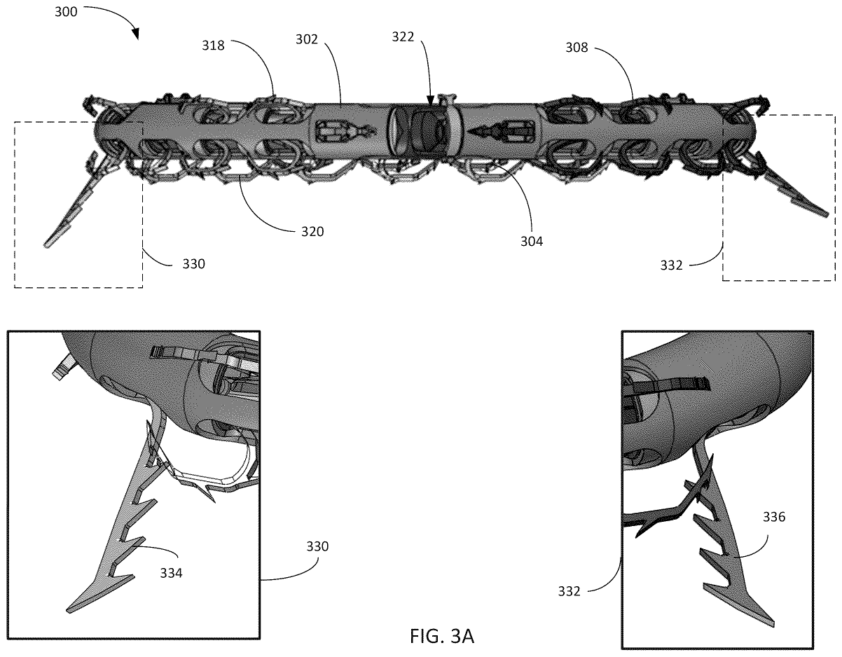

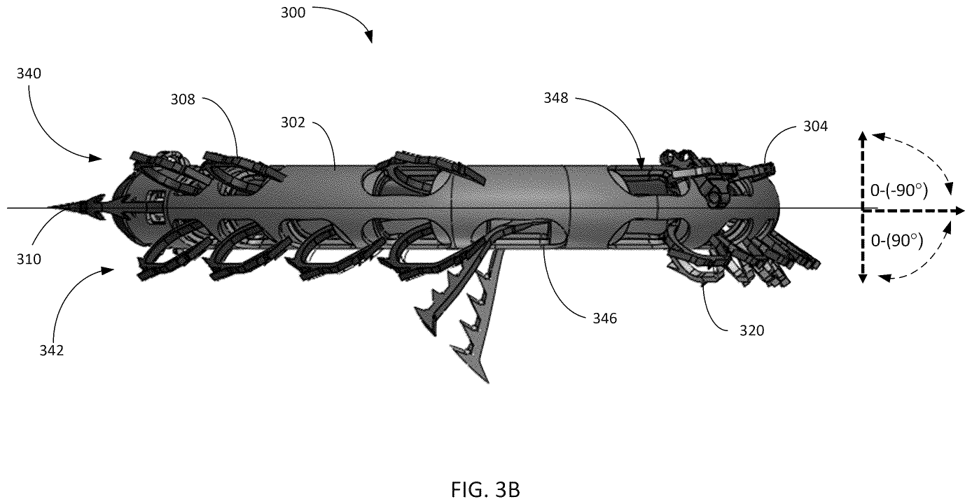

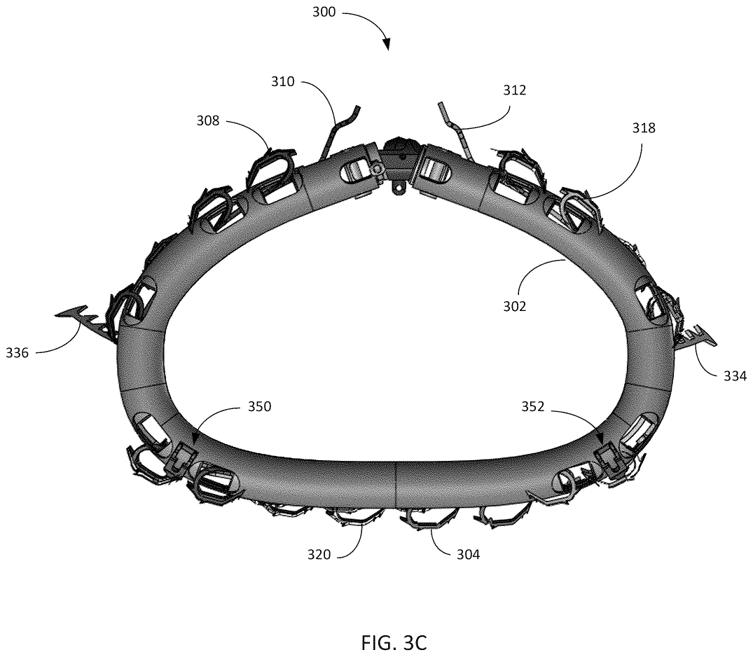

Referring now to FIGS. 3A-3C, side and top views of an annuloplasty device having anterior and posterior anchors, each with atrial and ventricular anchors, are depicted. Annuloplasty device 300, which may be the same as or similar to annuloplasty device 200 of FIG. 2, may include tube 302 as well as anterior anchors 320 and 304 and posterior anchors 318 and 308. Anterior anchors 320 and 304 and posterior anchors 318 and 308 may extend through windows of tube 302 in their deployed positions. Each end of tube 302 may be secured together using closure 322.

As shown in views 330 and 332 of FIG. 3A, anterior anchor 320 may include anchor 334 and anterior anchor 304 may include anchor 336 which each may have a generally linear, straight, and/or non-curved shape. Commissure anchors 334 and 336 may have one side that is serrated for engaging tissue of the native valve. Commissure anchors 334 and 336 may positioned along annuloplasty device 300 such that, when annuloplasty device is delivered to the native valve site (e.g., the mitral valve), anchors 334 and 336 are oriented to be inserted into and/or engage commissures of the native valve. In one example, anchors 334 and 336, despite extending from anterior anchor 320 and anterior anchor 304, respectively, may be positioned adjacent to and may extend into and engage posterior tissue of the native valve site. In this configuration, anchors 334 and 336 may work together with anchors of anterior anchor 320 and anterior anchor 304 positioned to engage anterior tissue of the native valve site to reduce the anterior-posterior distance and maintain (e.g., lock) the reduced anterior-posterior distance.

Referring now to FIG. 3B, a side view of annuloplasty ring 300 is illustrated. As shown in FIG. 3B, tube 302 may include ventricular windows 346 and atrial windows 348. Ventricular windows may be oriented towards a ventricular side of annuloplasty device 300 such that anchors (e.g., anchors 340) extending through ventricular windows 346 may be oriented to extend into ventricular tissue of the native valve and anchors (e.g., anchors 342) extending through atrial windows 348 may be positioned to extend through atrial tissue of the native valve.

Posterior anchor 308 may include atrial anchors extending from an annuloplasty device 300 at an angle of 0 to −45 degree from the centerline illustrated in FIG. 3B. In one example, atrial anchors of posterior anchor 308 may be positioned at an angle or −20 degrees from the centerline annuloplasty device 300. Posterior anchor 308 may further include ventricular anchors extending from a centerline of annuloplasty device 300 at an angle of 0 to 90 degrees. For example, the ventricular anchors of posterior anchor 308 may extend from the centerline of the annuloplasty device 300 at an angle of 45 degrees. Locking anchor 310 of posterior anchor 308 may extent from angle of 0 degrees from the centerline of annuloplasty device 300.

Atrial anchors of anterior anchor 304 may extend from centerline of annuloplasty device 300 from an angle between 0 and −45 degrees. For example, atrial anchors of anterior anchor 304 may extend from the centerline of annuloplasty device 300 at an angle of-20 degrees. Ventricular anchors of anterior anchor 304 and anterior anchor 320 may extend from the centerline of the annuloplasty device at an angle of 0 to 90 degrees. Ventricular anchors of anterior anchors 304 and 320 may be axially aligned with respect to the tube, may be offset from one another, and/or may have a difference in angle from the centerline of at least 5 degrees to avoid interfering with one another. For example, ventricular anchors or anterior anchor 304 may extend at 35 degrees from the centerline and ventricular anchors of anterior anchor 320 may extend at 55 degrees from the centerline of annuloplasty device 300.

Referring now to FIG. 3C, a top-down view of annuloplasty ring 300 is illustrated. As shown in FIG. 3C, anterior anchor 304 may include commissure anchor 336 and anterior anchor 320 include commissure anchor 334 may extend out of anterior windows of tube 302. Additionally, posterior anchor 308 including locking anchor 310 and posterior anchor 318 including locking anchor 312 may extend out posterior windows of tube 202. Docks 350 and 352 may be positioned on top the anterior side of tube 302 and may be designed to connect to a track anchor system of the catheter system, which may be used for aligning annuloplasty device 300 with the native valve.

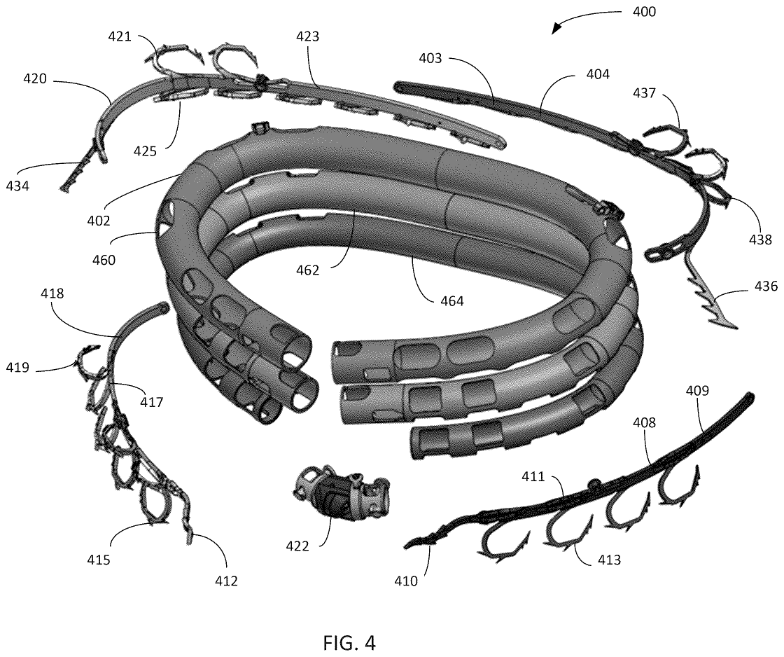

Referring now to FIG. 4, an exploded perspective view of an annuloplasty device having two posterior anchors and two anterior anchors is illustrated. Annuloplasty device 400 may be the same as or similar to annuloplasty device 300 of FIG. 3. As shown in FIG. 4, tube 402 may include multiple layers such as outer tube 460, intermediate tube 462, and inner tube 464. Outer tube 460 may be a polyester tube and/or cover for example and/or may encourage tissue ingrowth. Intermediate tube 462 may be a hollow ring tube and may form a ring shape. Inner tube 464 may be a laser cut polymer, for example, and/or may reduce friction between anchors and/or anchor components when such components move within tube 402. Each of outer tube 460, intermediate tube 462, and inner tube 464 may include several anterior and posterior windows and/or other windows (e.g., locking windows) for selectively deploying the anchors.

Annuloplasty device 400 may include four anchors, two anterior and two posterior. For example, anterior anchor 420 and anterior anchor 404 may be positioned within an interior of an anterior portion of tube 402. Anterior anchor 420 may include elongated base 423 which may be flat in shape and from which atrial anchors 421 and ventricular anchors 425 may extend from as well as commissure anchor 434. Similarly, anterior anchor 404 may include elongated base 403 which may have a similar shape as elongated base 423 and from which atrial anchors and ventricular anchors 438 may extend as well as commissure anchor 436. Atrial anchors 437 and 421 and ventricular anchors 438 and 435 may be curved in shape and may be serrated. In one example, atrial anchor 437, atrial anchor 421, ventricular anchor 438, and/or ventricular anchor 435 may be the same shape. The shape of commissure anchors 434 and 436 may be generally linear, straight, or non-curved. In one example, commissure anchors 434 and 436 may be the same shape.

Commissure anchors 434 and 436, despite extending from anterior anchor 420 and anterior anchor 404, respectively, may be positioned adjacent to and may extend into and engage posterior tissue of the native valve site. In this configuration, anchors 434 and 436 may work together with anchors of anterior anchor 420 and anterior anchor 404 positioned to engage anterior tissue of the native valve site (e.g., atrial anchors 421 and 437 and ventricular anchors 425 and 438) to reduce the anterior-posterior distance and maintain (e.g., lock) the reduced anterior-posterior distance.

Posterior anchors 418 and 408 may be positioned within a posterior portion of tube 402. Interior anchor 408 may include elongated base 409 which may be flat in shape and from which atrial anchors 411 and ventricular anchors 413 may extend from along with locking anchor 410. Similarly, interior anchor 418 may include elongated base 417 having a flat shape and atrial anchors 419 and ventricular anchors as well as locking anchor 412 that extend therefrom. Atrial anchors 419 and 411 and ventricular anchors 415 and 413 may have a curved shape with a serrated edge. The shape of locking anchors 412 and 410 may be generally linear, straight, or non-curved. In one example, atrial anchor 419, atrial anchor 411, ventricular anchor 415, and/or ventricular anchor 413 may have the same shape. Alternatively, or additionally, locking anchor 412 and locking anchor 410 may have the same shape.

Closure 422 may be positioned into ends of tube 402 and may be secured to each end of tube 402. For example, closure 422 may be the same as or similar to the closure device described in U.S. Pat. No. 11,576,779, the entire contents of which are incorporated herein by reference. Closure 422 may be lock having a primary engagement for engaging each end of the annuloplasty device to form an annular shape and a secondary engagement, different from the primary engagement selectively disengage ends of the annuloplasty device. For example, the primary engagement may be a snapping engagement and the secondary engagement may be formed from pin that may be removed to separate ends of the annuloplasty device. While three layers of tube 402, two posterior anchors, and two anterior anchors and illustrated, it will be one understood of one of ordinary skill in the art that any number of layers and/or anchors may be used.

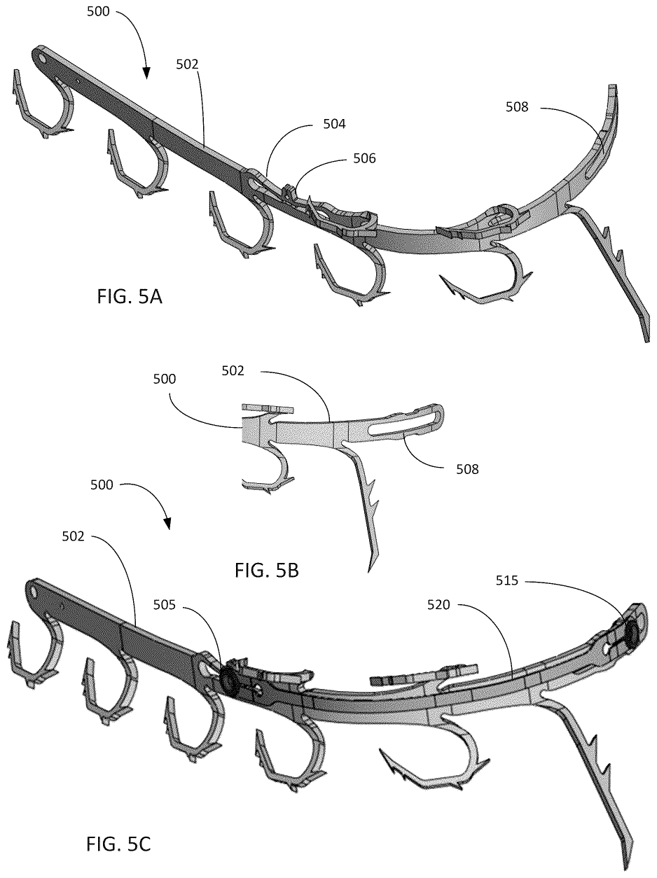

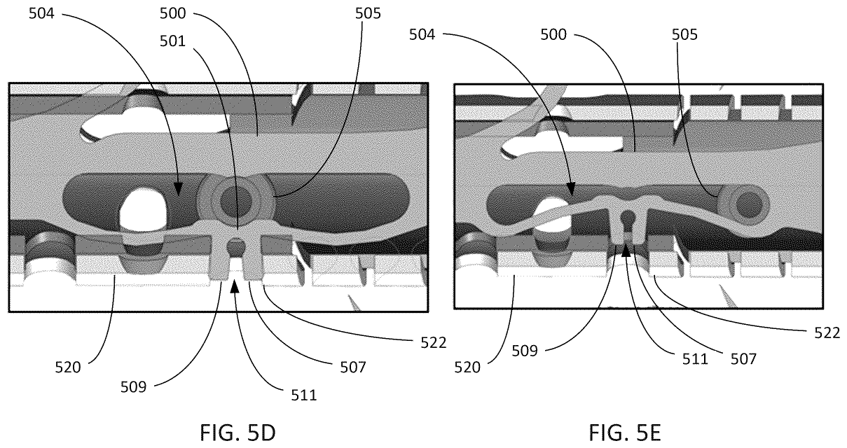

Referring now to FIG. 5A-5E perspective views and side views of an anterior anchor, pulley, and pulley activator of an annuloplasty device are illustrated. As shown in FIGS. 5A and 5B, anterior anchor 500 may include elongated base 502, which may be a flat and/or curved structure having several ventricular anchors (e.g., five) and atrial anchors (e.g., two) extending from elongated base 502. Elongated base 502 may include void 504 and void 508. Void 504 may be biased to reduce in size. Protrusion 506 may be positioned adjacent to void 504 and may move as void 504 increases and decreases in size.

Referring now to FIG. 5C, anterior anchor 500 may include pulley 505 positioned within void 504 and pulley 515 positioned within void 508. Pulley 505 and pulley 515 may be circular structures that move within and are guided by voids 504 and 508. Pulley 505 and pulley 515 may be connected by pulley activator 520, which may be in mechanical communication with each of pulley 505 and pulley 515 as with as the delivery handle positioned extracorporeally and may cause pulley 505 and pulley 515 to move together. Pulley 515 and/or pulley 505 may be engaged with a cable (e.g., suture, wire, cable, etc.) that may extend through the elongated catheter to the delivery handle and may be moved by the delivery handle to move pulleys 505 and 515 within voids 504 and 508 to transition anterior anchor 500 from a constrained position to a deployed position.

Referring now to FIGS. 5D and 5E, void 504 is illustrated with a reduced size in FIG. 5E as compared to FIG. 5D. As shown in FIG. 5D, pulley may be positioned in the middle of void 504 to cause void 504 to open, thereby extending protrusions 509 and 507 outward (e.g., away from void 504. Protrusions 509 and 507 may be adjacent to void 504 and may include void 511 separating protrusion 509 and 507. Void 504 may include one or more indention (e.g., indentation 501) such that pulley rests in indentation 501. When pulley 505 is in indentation 501 void 504 is caused to open thereby causing protrusions 507 and 509 to extend into locking window 522 of tube 520. With protrusions 507 and 509 in locking window 522, anterior anchor 500 may be prevented from moving with respect to tube 520.

As shown in FIG. 5E, to move anterior anchor 500 with respect to tube 520, the delivery handle may be actuated or otherwise manipulated to advance pulley 505 to the left or the right to permit void 504 to reduce in size. As void 504 reduces in size, protrusions 509 and 507 may be retracted upward and void 511 may be reduced in size as protrusions 509 and 507 are retracted upward, causing protrusions 507 and 509 to exit locking window 522. With protrusions 507 and 509 removed from locking window, anterior anchor 500 may move with respect to tube 520.

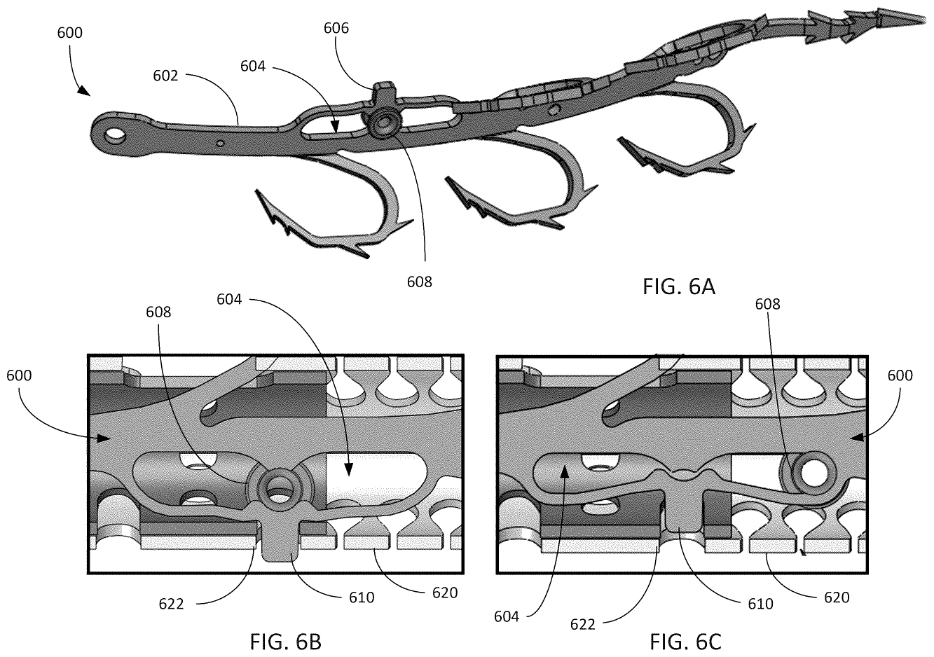

Referring now to FIGS. 6A-6C, perspective views and side views of a posterior anchor and pulley of an annuloplasty device are illustrated. Posterior anchor 600 may include elongated base 602 which may be a flat and/or curved structure from which multiple ventricular anchors (e.g. three) and multiple atrial anchors (e.g., two) extend as well as a locking anchor. Elongated base 602 may further include void 604 which may be biased to reduce in size and may guide pulley 608 within void 604. Protrusion 610 may be positioned adjacent to void 604 and may extend away from void 604 when void 604 increases in size and retract toward void 604 when void is reduced in size.

When pulley 608 is positioned in the middle of void 604, pulley 608 may cause void 604 to expand, causing protrusion 610 to extend into locking window 622 of tube 620. As shown in FIGS. 6B and 6C, the delivery handle may connect to pulley 608 (e.g., via a cable, suture, wire, etc.) and may cause pulley 608 to move to right or the left to cause void 604 to reduce in size. When void 604 reduces in size, protrusion 610 is retracted towards void 604 and thus exits locking window 610, permitting posterior anchor 600 to move with respect to tube 620.

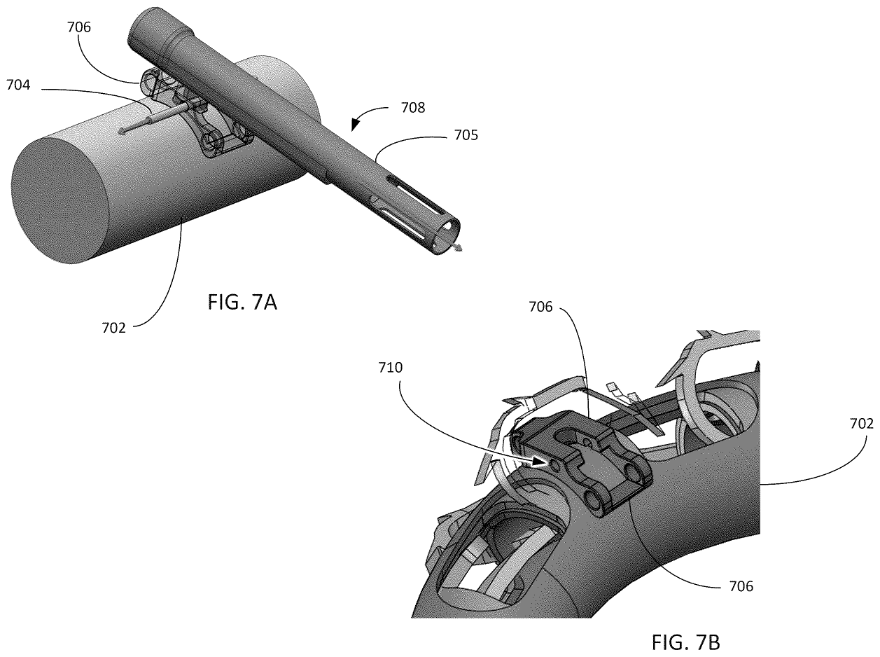

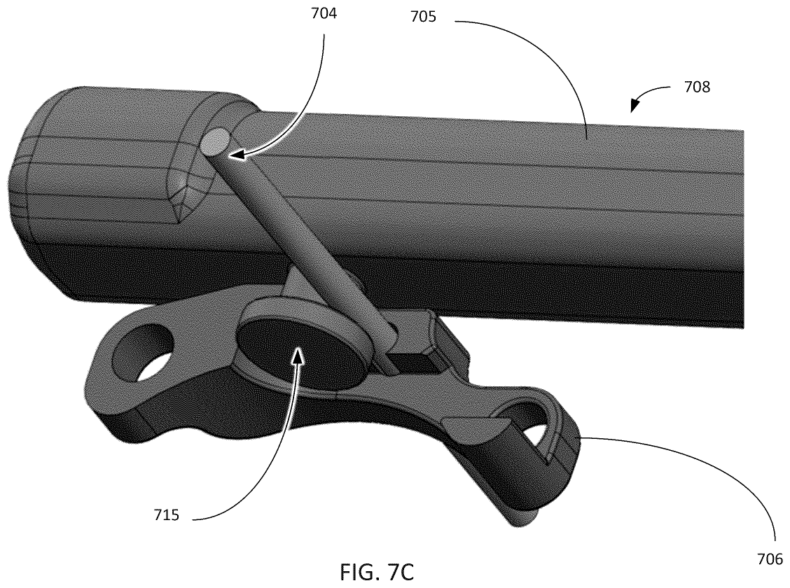

Referring now to FIGS. 7A-7C, perspective views and cut-away views of a track anchor and a dock of an annuloplasty device are illustrated. As shown in FIG. 7A, tube 702 of a annuloplasty device which may be the same as or similar to annuloplasty ring 300 of FIG. 3A, may include dock 706 which may connect to track anchor system 708 and specifically sleeve 705 of track anchor system 708, which may deliver track anchors for aligning the annuloplasty device with the native valve site. Release cable 704 may be inserted into dock 706 and through a portion of track anchor system 708 to secure track anchor system 708 to dock 706.

FIG. 7B illustrates dock 706 connected to an outer surface of tube 705. Dock 706 may include through holes 710 for receiving a release cable to secure the track anchor system to dock 706. Release cable may be connected to the delivery handle and may be manipulated by the delivery handle. In another example, multiple release cables may be used. Referring now to FIG. 7C, a cut-away perspective view of dock 706 is illustrated showing protrusion 715 which may have disk shape offset from sleeve 705 of anchor track system 708 such that release cable 704 may be inserted between protrusion 715 and sleeve 705 to prevent protrusion 715 from being released from dock 706. When release able 704 is removed from dock 706, sleeve 705 may be removed from dock 706.

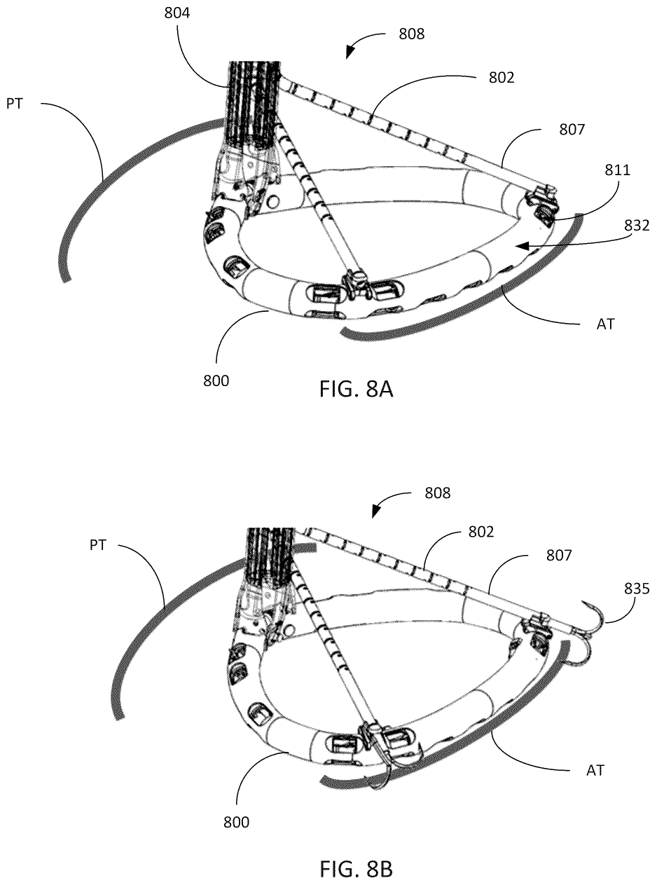

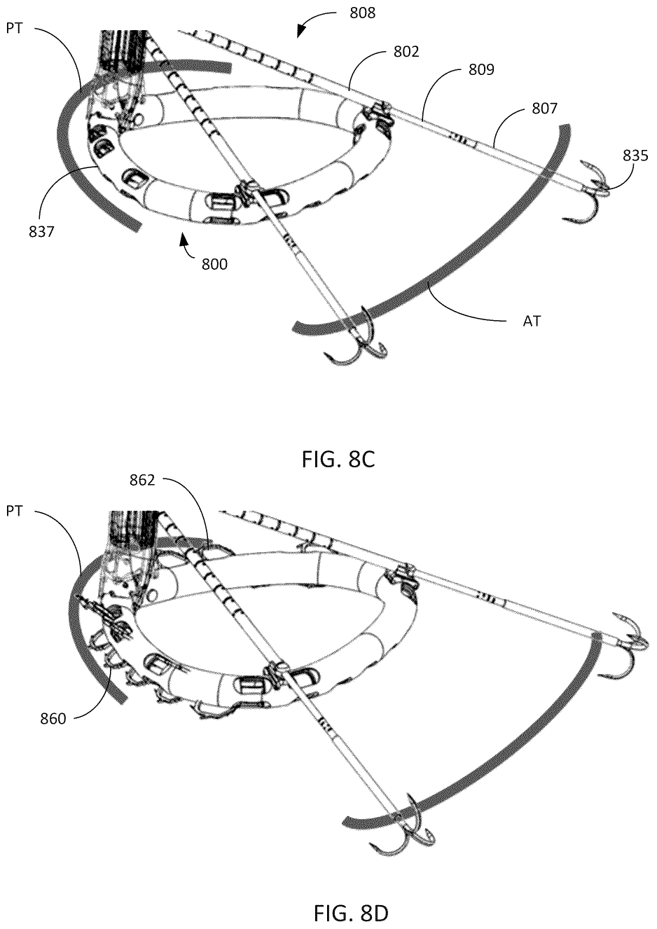

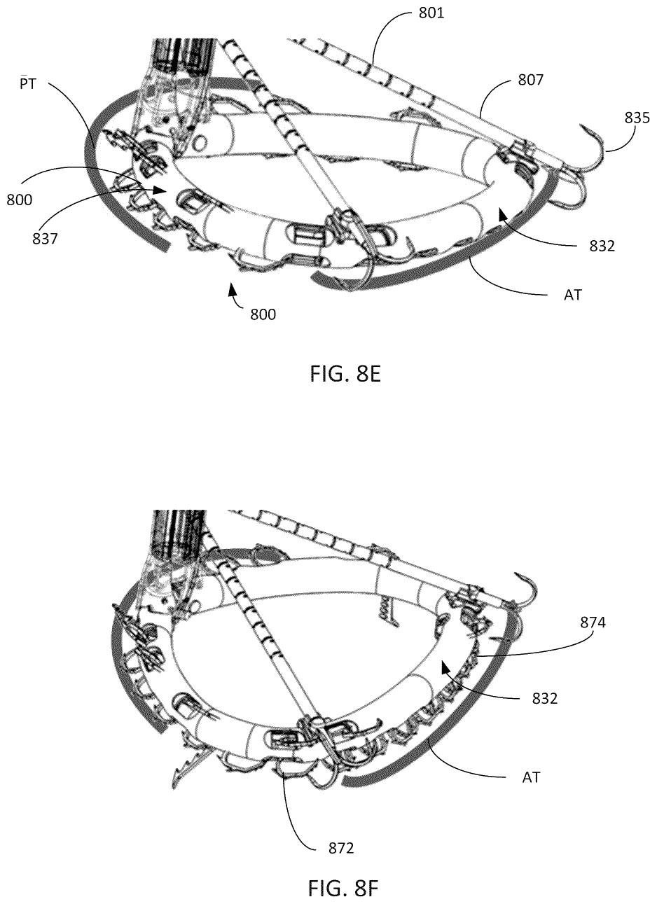

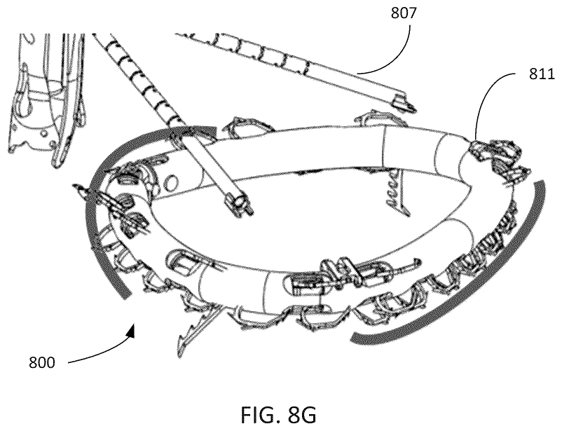

Referring now to FIGS. 8A-8G, perspective views of an annuloplasty device deploying track anchors, posterior anchors, and anterior anchors for reducing an anterior-posterior distance of a native valve are illustrated. As shown in FIG. 8A, annuloplasty device 800, which may be the same as or similar to annuloplasty device 300 of FIG. 3A, may be delivered to a native valve site (e. g, mitral valve site or tricuspid valve site), and anterior side 832 of annuloplasty device 800 having anterior anchors for engaging anterior tissue of the native valve site (e.g., a portion of the native valve from which anterior leaflets extend) may be aligned with the anterior tissue via distal end 804 of the delivery catheter and track anchor system 808. Track anchor system 808 may include two of such hollow cannulas (e.g., cannulas 802) that may be connected to sleeves (e.g. sleeves 807), which may be releasably connected to the tube of annuloplasty device 800 at docks 811, as shown in FIGS. 7A-7C. In one example, track anchor system 808 may be similar to the track anchor system described in more detail in U.S. Pat. No. 11,857,418, the entire contents of which are incorporated herein by reference.

Anchor track system 808, which may include cannulas 802 from distal end 804 of the delivery catheter and connect sleeves (e.g. sleeve 807) which may connect to the docks (e.g., docks 811) of annuloplasty device 800 may be used to align annuloplasty device 800 with the native valve site, and specifically align the anterior portion 832 of annuloplasty device 800 with anterior tissue AT of the native valve site. Once anterior portion 832 is adjacent to anterior tissue AT of the native valve site, the delivery handle may be manipulated to cause track anchors 835 to traverse track anchor system 808 and exit a distal end of sleeve 807 to insert a portion of track anchors 835 into the native anterior valve tissue, as shown in FIG. 8B. For example, two track anchors, one from each sleeve of track anchor system 808 may extend into tissue of the native heart valve.

Referring now to FIG. 8C, once track anchors 835 are deployed into anterior tissue AT, distal end 804 may move annuloplasty device 800 to align posterior portion 837 of annuloplasty device 800 with posterior tissue PT. As annuloplasty device 800 is moved away from track anchors 835, cables 809 connected at one end of track anchors 835 may maintain a connection between track anchors 835 and track anchor system 808. Once annuloplasty device 800 is positioned adjacent to posterior tissue PT, posterior anchors 860 and 862 may be deployed into the posterior tissue of the native valve, as shown in FIG. 8D. Posterior anchors may be the same as or similar to posterior anchors 418 and 408 of FIG. 4, for example. Posterior anchors 860 and 862 may be deployed sequentially using separate actuators on the delivery handle or may be deployed at the same time using a single actuator on the delivery handle.

Referring now to FIG. 8E, cable 809 (not shown) may be retracted into cannula 802 to pull annuloplasty device 800 back towards anterior tissue AT to reduce the distance between posterior tissue PT and anterior tissue AT thereby causing posterior portion 837 of annuloplasty device 800 to be adjacent to anterior tissue PT and anterior portion 832 to be adjacent to anterior tissue AT. As shown in FIG. 8F, once anterior portion 832 of annuloplasty device 800 is adjacent anterior tissue AT, anterior anchors 872 and 874 may be deployed and extended into anterior tissue AT to secure anterior portion 832 into anterior tissue AT. Anterior anchors 872 and 874 may be the same as or similar to anterior anchors 420 and 404 of FIG. 4. Anterior anchors 872 and 874 may be deployed sequentially using separate actuators on the delivery handle or may be deployed at the same time using a single actuator on the delivery handle. In one example, anchors 888 and 889 may extend from anterior anchor 872 and anterior anchor 874, respectively, and may be positioned adjacent to and may extend into and engage posterior tissue of the native valve site. In this configuration, anchors 888 and 889 may work together with anchors of anterior anchor 874 and anterior anchor 889 positioned to engage anterior tissue of the native valve site to reduce the anterior-posterior distance and maintain (e.g., lock) the reduced anterior-posterior distance illustrated in FIG. 8F.

Referring now to FIG. 8G, once posterior and anterior anchors are deployed from annuloplasty device 800, the track anchors may be retracted into sleeve 807 and each sleeve 807 may be disconnected from docks 811 by manipulating the delivery device to remove the release cable, thereby disconnecting annuloplasty device 800 from the distal end of the delivery catheter. Once annuloplasty device 800 is released from the delivery catheter, the delivery catheter may be retracted and removed from the patient, leaving the annuloplasty device implanted at the native valve site.

The foregoing description of illustrative embodiments has been presented for purposes of illustration and of description. It is not intended to be exhaustive or limiting with respect to the precise form disclosed, and modifications and variations are possible in light of the above teachings or may be acquired from practice of the disclosed embodiments. It is intended that the scope of the invention be defined by the claims appended hereto and their equivalents.

What is claimed is:

1. A method for implanting an annuloplasty device, the method comprising:
delivering an annuloplasty device in an elongated shape to a native heart valve of a patient, the annuloplasty device comprising (1) a tube having an inner lumen, a first plurality of windows, and a second plurality of windows, (2) at least one anterior anchor disposed in the tube and comprising (i) a first elongated base, (ii) a plurality of first anchors extending from the first elongated base, each first anchor of the plurality of first anchors having a first shape, and (iii) at least one second anchor extending from the first elongated base and having a second shape different than the first shape, and (3) at least one posterior anchor disposed in the tube comprising (x) a second elongated base, (y) a plurality of third anchors extending from the second elongated base, each third anchor of the plurality of third anchors having a third shape, and (z) at least one fourth anchor extending from the second elongated base and having a fourth shape different than the third shape;
causing the annuloplasty device to transition from the elongated shape to an annular shape and orientation with respect to the native heart valve such that the first plurality of windows is oriented toward anterior tissue of the native heart valve and the second plurality of windows is oriented toward posterior tissue of the native heart valve,
causing the plurality of third anchors to extend through at least some of the second plurality of windows to anchor into the posterior tissue of the native heart valve;
reducing a distance between the posterior tissue of the native heart valve and the anterior tissue of the native heart valve; and
causing the plurality of first anchors to extend through at least some of the first plurality of windows to anchor into the anterior tissue of the native heart valve.

2. The method of claim 1, wherein the at least one posterior anchor further comprises at least one fifth anchor extending from the second elongated base and having a fifth shape different than the fourth shape.

3. The method of claim 1, wherein the at least one anterior anchor further comprises at least one fifth anchor extending from the first elongated base and having a fifth shape different than the second shape.

4. The method of claim 1, wherein the at least one second anchor extends into posterior tissue of the native valve when the plurality of first anchors extend through at least some of the first plurality of windows.

5. The method of claim 1, wherein the at least one fourth anchor extends into posterior tissue of the native heart valve when the plurality of third anchors extend through at least some of the second plurality of windows.

6. The method of claim 1, wherein the tube further comprises a locking window configured to prevent the plurality of first anchors from extending through the at least some of the first plurality of windows.

7. The method of claim 6, wherein the at least one anterior anchor comprises a lock having a first engagement configured to engage and selectively disengage the locking window.

8. The method of claim 1, wherein the at least one second anchor is substantially linear and is positioned at an end of the first elongated base.

9. The method of claim 1, wherein the at least one fourth anchor is substantially linear and is positioned at an end of the second elongated base.

10. The method of claim 1, wherein a delivery catheter is used to deliver the annuloplasty device and the delivery catheter is also used to reduce the distance between the posterior tissue of the native heart valve and the anterior tissue of the native heart valve.

11. A method for implanting an annuloplasty device, the method comprising:
delivering an annuloplasty device in an elongated shape to a native heart valve of a patient, the annuloplasty device comprising (1) a tube having an inner lumen, a first plurality of windows, and a second plurality of windows, (2) at least one anterior anchor disposed in the tube and comprising (i) a first elongated base, (ii) a plurality of first anchors extending from the first elongated base at a first orientation, and (iii) a plurality of second anchors extending from the first elongated base at a second orientation different than the first orientation, and (3) at least one posterior anchor disposed in the tube comprising (x) a second elongated base, (y) a plurality of third anchors extending from the second elongated base at a third orientation, and (z) a plurality of fourth anchors extending from the second elongated base at a fourth orientation different than the third orientation;
causing the annuloplasty device to transition from the elongated shape to an annular shape and orientation with respect to the native heart valve such that the first plurality of windows is oriented toward anterior tissue of the native heart valve and the second plurality of windows is oriented toward posterior tissue of the native heart valve,
causing the plurality of third anchors and the plurality of fourth anchors to extend through at least some of the second plurality of windows to anchor into the posterior tissue of the native heart valve such that the plurality of third anchors are oriented in a first ventricular direction and the plurality of fourth anchors are oriented in a first atrial direction;
reducing a distance between the posterior tissue of the native heart valve and the anterior tissue of the native heart valve; and
causing the plurality of first anchors and the plurality of second anchors to extend through at least some of the first plurality of windows to anchor into the anterior tissue of the native heart valve such that plurality of first anchors are oriented in a second ventricular direction and the plurality of second anchors are oriented in a second atrial direction.

12. The method of claim 11, wherein the at least one posterior anchor further comprises at least one fifth anchor extending from the second elongated base and having a shape different than respective shapes of the plurality of third anchors and the plurality of fourth anchors.

13. The method of claim 12, wherein the at least one fifth anchor extends into posterior tissue of the native valve when the plurality of third anchors and the plurality of fourth anchors extend through at least some of the second plurality of windows.

14. The method of claim 12, wherein the at least one fifth anchor is substantially linear and is positioned at an end of the second elongated base.

15. The method of claim 11, wherein the at least one anterior anchor further comprises at least one fifth anchor extending from the first elongated base and having a shape different than respective shapes of the plurality of first anchors and the plurality of second anchors.

16. The method of claim 15, wherein the at least one fifth anchor extends into posterior tissue of the native valve when the plurality of first anchors and plurality of second anchors extend through at least some of the first plurality of windows.

17. The method of claim 15, wherein the at least one fifth anchor is substantially linear and is positioned at an end of the first elongated base.

18. The method of claim 11, wherein the tube further comprises a locking window configured to prevent the plurality of first anchors and the plurality of second anchors from extending through the at least some of the first plurality of windows.

19. The method of claim 18, wherein the at least one anterior anchor comprises a lock having a first engagement configured to engage and selectively disengage the locking window.

20. The method of claim 11, wherein a delivery catheter is used to deliver the annuloplasty device and the delivery catheter is also used to reduce the distance between the posterior tissue of the native heart valve and the anterior tissue of the native heart valve.
